(19) United States
(12) Patent Application Publication
(10) Pub. No.: US 2026/0248364 A1
(43) Pub. Date: Aug. 27, 2026

(54) REMOTE IMAGING SYSTEM FOR MEDICAL ENDOSCOPIC SYSTEM FOR VIEWING A TARGET

(71) Applicant: AXESS VISION TECHNOLOGY, JOUE LES TOURS (FR)

(72) Inventors: Xavier DIEUDONNÉ, MONTS (FR); Emmanuel HALLAUER, SACHE (FR)

(73) Assignee: AXESS VISION TECHNOLOGY, JOUE LES TOURS (FR)

(21) Appl. No.: 19/156,855

(22) PCT Filed: Dec. 8, 2023
(86) PCT No.: PCT/FR2023/051963

§ 371 Date: Aug. 14, 2025

(30) Foreign Application Priority Data
Dec. 8, 2022 (FR) FR2212973

(51) Int. Cl. A61B 1/00; A61B 1/07

(52) U.S. Cl. CPC A61B 1/00013; A61B 1/00045; A61B 1/00117; A61B 1/00126; A61B 1/00167; A61B 1/07

(57) ABSTRACT

The invention relates to an imaging system including: a medical endoscopic system including an insertion instrument provided with at least one multicore optical fiber, the distal end of which is located at the distal head of the insertion instrument, the proximal end of the multicore optical fiber being provided with an optical connector; a device for acquiring and processing images including: at least a first illumination source configured to deliver a light beam in at least a first wavelength spectrum, to the multicore optical fiber via the optical connector; at least one imaging sensor configured to receive a light beam coming from at least the proximal end of the multicore optical fiber; and an imaging processor connected to the imaging system and configured to form images of the target.

### TECHNICAL FIELD

This invention relates to the technical field of imaging systems implemented in the context of medical endoscopic systems in the general sense, used to access the inside of a body such as a cavity or a canal for example, and more specifically pertains to endoscopic medical systems: medical catheters and medical endoscopes.

The medical endoscopic system implemented in the context of this invention has particularly advantageous applications in making it possible to access the inner surface of a hollow organ, a cavity or a natural or artificial duct of the human body for the purpose of carrying out various operations for therapeutic, surgical or diagnostic purposes, and which can be used in the field of urinary tracts, gastro-intestinal tracts, the respiratory system, the cardiovascular system, the trachea, the sinus cavity, the female reproductive system, the abdominal cavity or any other part of the human body to be explored via a natural or artificial pathway.

### PRIOR ART

Conventionally, a medical endoscopic system of medical catheter or medical endoscope type includes a control handle to which is attached an insertion tube having, opposite its part attached to the control handle, a distal head. This insertion tube has a greater or lesser length and flexibility so as to be able to be introduced into a natural or artificial pathway for the purpose of carrying out various operations or functions for therapeutic, surgical or diagnostic purposes. Note that such an endoscopic system is designed to have the smallest possible section to be able to access access pathways of limited through section.

For a medical endoscopic system of endoscope type, the distal head is equipped in particular with a viewing system making it possible to examine the organ, cavity or duct of the human body. Upstream of this distal head, the insertion tube includes a flexible or articulating part formed of articulated vertebra allowing the distal head to be pivoted. This medical endoscope is intended to be connected to a medical electronic device including a unit for processing the image signals delivered by the viewing system of the endoscope. The images taken are viewed on a screen of this device or on a remote screen connected to this device.

The viewing system mounted on the distal part of the tube includes a camera associated or not associated with one or more light sources such as light-emitting diodes. The camera, or even the light sources are electrically connected to electrical components located in the handle or in the medical device. According to the exemplary embodiment described by patent application US 2022/0160218, the camera and light sources located at the distal part of the insertion tube are connected to the electrical components located in the handle. Note that a medical endoscopic system is generally used in an environment in which various electrical equipment items are in operation such as electrosurgical scalpels, X-ray devices, scanners or screens, liable to affect the operation of the camera and/or the signal delivered by the camera. Moreover, in the case of a disposable endoscopic system, the light sources and the camera are disposed of as waste.

In addition, these electronic components are electronic waste requiring recycling, increasing the cost of such a system.

There is also known from patent US 11 061 185 a medical endoscopic system including a multicore optical fiber composed of a large number of cores separated by a matrix and housed in a common sheath. This multicore optical fiber receives the radiation from a target at its distal end and transmits the radiation over its entire length to deliver the radiation, at its proximal part, to an imaging sensor. The medical endoscopic system also includes an illumination source delivering, at the proximal end of an illuminating optical fiber, a light beam departing from the distal end of the illuminating fiber to illuminate the target.

Such a medical endoscopic system has a sensitivity to electromagnetic disruptions of zero by comparison with other medical endoscopic systems.

However, this medical endoscope requires the use of a multicore optical fiber and illumination for the purpose of obtaining a quality image of the target. It ensues that such an endoscopic system does not have a section allowing it to access access pathways of limited through section. Moreover, the illumination is remote from the distal end of the multicore optical fiber such that the area of the target observed by the sensor may be poorly illuminated.

### SUMMARY OF THE INVENTION

The subject of the invention has the aim of remedying the drawbacks of the prior art by making provision for an imaging system including a medical endoscopic system insensitive to electromagnetic disturbances and having a limited through section while also obtaining an optimized target image quality.

Another subject of the invention is to make provision for an imaging system including a medical endoscopic system having a ratio of its through section to the image resolution that is as low as possible in order to obtain an optimized image target image quality for a reduced through section.

Another subject of the invention is to make provision for an imaging system designed to have a reduced manufacturing cost while limiting waste in the case of a disposable endoscopic system.

To achieve these objectives, the imaging system for a medical endoscopic system for viewing a target includes:
a medical endoscopic system including an insertion instrument terminating, opposite a proximal part, in a distal head, this insertion instrument being provided with at least one multicore optical fiber having a distal end and a proximal end, the distal end of the multicore optical fiber being located at the distal head of the insertion instrument while the multicore optical fiber is provided with an optical connector at its proximal end and extends at least all the way to the proximal part of the insertion instrument;
a device for acquiring and processing images including:
at least a first illumination source configured to deliver a light beam in at least a first wavelength spectrum, to the multicore optical fiber via the optical connector;
at least one imaging sensor configured to receive a light beam coming from at least the proximal end of the multicore optical fiber;
an imaging processor connected to the imaging sensor and configured to form images of the target.

According to an exemplary embodiment, the medical endoscopic system includes a single multicore optical fiber while the device for acquiring and processing images includes:
an illumination source configured to deliver a light beam in at least a first wavelength spectrum, to the single multicore optical fiber via the optical connector;
a single imaging sensor configured to receive a light beam coming from the proximal end of the single multicore optical fiber.

According to another exemplary implementation:
the endoscopic system includes:
a first multicore optical fiber and a second multicore optical fiber,
the device for acquiring and processing images includes:
an illumination source configured to deliver a light beam to the first multicore optical fiber,
a single imaging sensor configured to have a first area for receiving a light beam coming from the proximal end of the first multicore optical fiber and a second receiving area separate from the first receiving area, for receiving a light beam coming from the proximal end of the second multicore optical fiber.

According to another exemplary implementation:
the endoscopic system includes:
a first multicore optical fiber and a second multicore optical fiber,
the device for acquiring and processing images includes:
a first illumination source configured to deliver a light beam to the first multicore optical fiber,
a second illumination source configured to deliver a light beam to the second multicore optical fiber,
a single imaging sensor configured to have a first area for receiving a light beam coming from the proximal end of the first multicore optical fiber and a second receiving area separate from the first receiving area, for receiving a light beam coming from the proximal end of the second multicore optical fiber.

According to another exemplary implementation:
the endoscopic system includes:
a first multicore optical fiber and a second multicore optical fiber,
the device for acquiring and processing images includes:
an illumination source configured to deliver a light beam to the first multicore optical fiber,
a first imaging sensor configured to receive a light beam coming from the proximal end of the first multicore optical fiber,
a second imaging sensor configured to receive a light beam coming from the proximal end of the second multicore optical fiber.

According to another exemplary implementation:
the endoscopic system includes:
a first multicore optical fiber and a second multicore optical fiber,
the device for acquiring and processing images includes:
a first illumination source configured to deliver a light beam to the first multicore optical fiber,
at least a second illumination source configured to deliver a light beam to the second multicore optical fiber,
a first imaging sensor configured to receive a light beam coming from the proximal end of the first multicore optical fiber,
a second imaging sensor configured to receive a light beam coming from the proximal end of the second multicore optical fiber.

Advantageously:
the illumination source or sources are configured to deliver light beams in different wavelength spectra,
the imaging sensor or sensors are configured to acquire images of different wavelength spectra,
the imaging processor processes the images of different wavelength spectra to obtain a spectral super-resolution image.

According to an example, the imaging sensor or sensors are configured to acquire images of different wavelength spectra by means of colored filters, the colors of which correspond to the different wavelength spectra of the light beams.

For Example:
the illumination source or sources are configured to deliver light beams in the red, green and blue wavelength spectra;
the imaging sensor or sensors are configured to acquire images in the red, green and blue wavelength spectra;
the imaging processor processes the wavelength spectra images to obtain a contrasted or colored image.

For this example, making it possible to obtain a spectral super-resolution image, by way of illustration, the sensors are configured to acquire red, green and blue wavelength spectrum images by means of filters (for example arranged in a Bayer matrix) filtering certain wavelengths arriving at each individual pixel (an individual pixel includes a photosite). These filters can be microlenses. Also, these filters can be on lenses or on the sensor itself.

It appears that each individual pixel (sometimes known as sub-pixel) has an assigned red, green or blue color. Using an illumination in the red, green and blue wavelength spectra, one thus has individual pixels in the sensor which can be used according to the spectrum used for the illumination, which makes it possible to accurately locate the perceived variations. By recombining the images obtained by means of the different spectra, a super-resolution is obtained.

Moreover, the chromatic aberrations or the different absorbency of the tissues can in this example make new details appear, according to the spectra used.

According to another example:
the imaging processor controls the imaging sensor or sensors to acquire temporally-offset images,
the imaging processor processes the temporally-offset images to obtain a temporal super-resolution image.

This example takes into account the movements of the patient into whom the insertion instrument is inserted, movements which can be caused by the breathing of the patient. The temporally-offset images thus also have a spatial offset, making it possible, by processing, to obtain a super-resolution, for example by image recombination.

In this example, there are no actuators used to displace the insertion instrument.

Advantageously, in this example, the speed of acquisition of the super-resolution images is greater than 24 images per second, which implies an acquisition speed of the individual images that is greater than a multiple of 24, or 24 times n with n the number of temporally-offset images which are used to obtain an image.

Alternatively, the acquisition speed may be greater than 24 images per second, which makes it possible to implement a temporal super-resolution at least for a part of the images, or even by reusing certain images. For this alternative, use may be made of augmentation techniques intended to add images obtained by image duplication or by combination of images obtained by acquisition.

Advantageously, the insertion instrument is static.

The term “static” should be understood to mean that the insertion instrument does not include any automatic means capable of displacing one or more elements of the insertion instrument, for example, it does not include any actuators.

This results in a simple device, which uses the respiratory movements of the patient for the temporal super-resolution.

According to another advantageous example:
the imaging processor controls the imaging sensor or sensors to acquire spatially-offset images having an area of overlap,
the imaging processor processes the spatially-offset images to obtain a spatial super-resolution image.

For example, the spatially-offset images with an area of overlap can be obtained using two networks in the multicore fiber or fibers, these two networks being offset with respect to one another.

This offset is implemented so that the two networks do not overlap with one another, i.e. to avoid receiving the same light information for two elements of the multicore fiber belonging to two different networks. There is nonetheless an area of overlap within the images obtained by the imaging sensors.

Preferably, by way of illustration, one acquires images spatially offset by one half fiber core along at least one axis of the image plane (generally denoted as the plane X, Y, here, each half fiber core belongs to a different one of said networks). For each half fiber core an image is obtained. Preferably, the pixels of the imaging sensor are of small dimensions, with at least 3 pixels of each color to obtain accurate colorimetry information. Due to the offset, a super-resolution is obtained.

It was found that the images can have a discontinuous (dotted) appearance, and it is possible to use artificial intelligence techniques, for example automated learning, to obtain a super-resolution that makes the dots disappear.

By way of information, the following artificial intelligence techniques and in particular automated learning techniques can be used:
ESRGAN: Enhanced Super-Resolution Generative Adversarial Networks (Xintao Wang et al., arXiv:1809.00219);
Accelerating the Super-Resolution Convolutional Neural Network (Chao Dong et al., arXiv:1608.00367).

According to a variant embodiment, the medical endoscopic system includes an illuminating optical fiber having a distal end and a proximal end, the distal end of the illuminating optical fiber being located at the distal head of the insertion instrument while the proximal end of the illuminating optical fiber is located at the proximal part of the insertion instrument while being provided with an optical connector via which is conveyed an illuminating light beam supplied by an illumination source.

Advantageously, the device for acquiring and processing images includes an optical separating system disposed on the optical path between the proximal end of a multicore optical fiber and an imaging sensor and reflecting in the direction of the proximal end of a multicore optical fiber, the light beam coming from an illumination source.

According to an embodiment, the medical endoscopic system includes as the insertion instrument an insertion tube terminating in a distal head and held at its opposite end by a control handle, the multicore optical fiber or fibers being mounted inside the insertion tube.

According to another embodiment, the medical endoscopic system includes as the insertion instrument a catheter including the multicore optical fiber or fibers.

### BRIEF DESCRIPTION OF THE DRAWINGS

FIG. 1 is an overall view of an example of an application of a remote imaging system for an endoscope as the medical endoscopic system for viewing a target.

FIG. 2 is an overall view of another example of an application of a remote imaging system for an endoscope as the medical endoscopic system for viewing a target.

FIG. 3 is an overall view of another example of an application of a remote imaging system for a catheter as the medical endoscopic system for viewing a target.

FIG. 4 schematically represents a first exemplary embodiment of a remote imaging system for a medical endoscopic system implementing a single multicore optical fiber, one light source and one imaging sensor.

FIG. 5 schematically represents another exemplary embodiment of a remote imaging system for a medical endoscopic system implementing two multicore optical fibers, one light source and one imaging sensor.

FIG. 6 schematically represents another exemplary embodiment of a remote imaging system for a medical endoscopic system implementing two multicore optical fibers, two light sources and one imaging sensor.

FIG. 7 schematically represents another exemplary embodiment of a remote imaging system for a medical endoscopic system implementing two multicore optical fibers, one light source and two imaging sensors.

FIG. 8 schematically represents another exemplary embodiment of a remote imaging system for a medical endoscopic system implementing two multicore optical fibers, two light sources and two imaging sensors.

FIG. 9 schematically represents the distal head of a medical endoscope including two multicore optical fibers and showing a detail of a multicore optical fiber.

FIG. 10 is a functional block diagram of an exemplary embodiment of a remote imaging system for a medical endoscopic system.

FIG. 11 schematically represents another exemplary embodiment of a remote imaging system for a medical endoscopic system implementing two multicore optical fibers, one light source and two imaging sensors as well as an additional illuminating fiber.

### DESCRIPTION OF THE EMBODIMENTS

As can be seen from the figures, the subject of the invention relates to an imaging system I for a medical endoscopic system 1 of endoscope or catheter type in the general sense designed to access the inside of a body such as a cavity or a canal for example. Conventionally, an endoscopic system 1 of endoscope or catheter type includes an insertion instrument 2 having a proximal part 2a and opposite it, a distal part forming a free end. The insertion instrument 2 thus terminates at its free end in a distal head 2b from which is viewed a target C in the general sense.

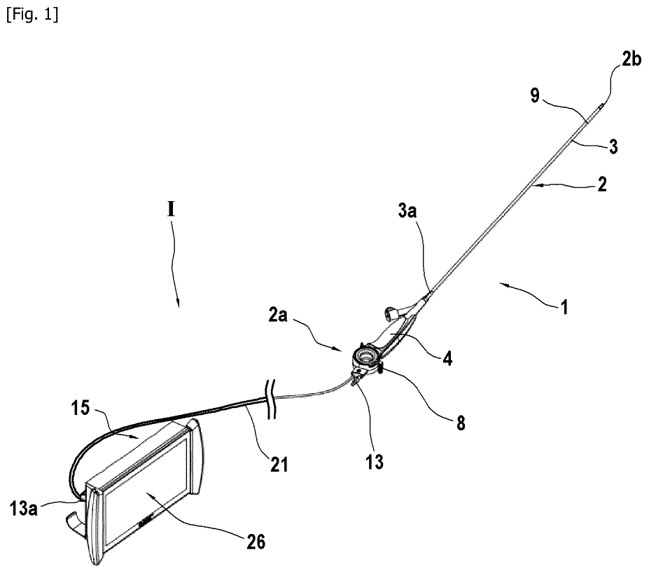

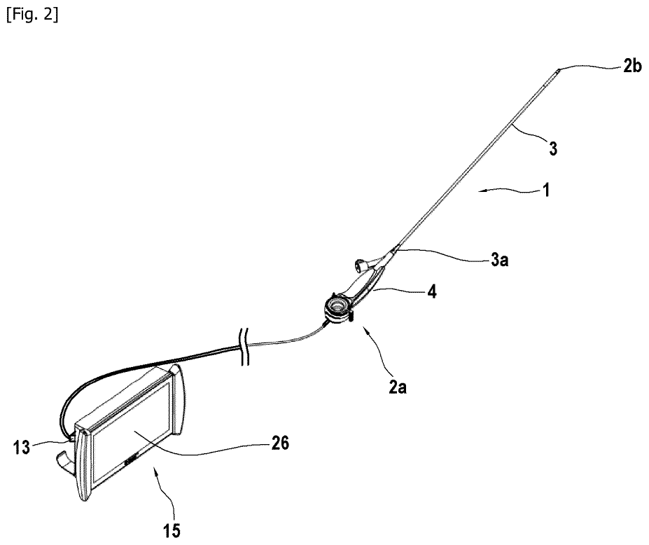

According to a mode of application for which the medical endoscopic system 1 is an endoscope (FIGS. 1, 2 and 9), the medical endoscopic system includes as the insertion instrument 2 an insertion tube 3 having a free end forming the distal head 2b and held at its opposite end by a control handle 4, all or part of which forms the proximal part 2a of the insertion instrument. The insertion tube 3 is temporarily or permanently attached to the control handle 4. In the example illustrated in FIGS. 1 and 2, the insertion tube 3 is engaged by its opposite end with its free end, in a housing of an endpiece 3a intended to be attached to the distal part of the control handle 4. This insertion tube 3, which has a greater or lesser length and flexibility, is intended to be introduced into a natural or artificial access pathway for the purpose of performing various operations or functions for therapeutic, surgical or diagnostic purposes.

The insertion tube 3 is made of a semi-rigid material such as for example thermoplastic elastomer (TPE). The insertion tube 3 has a length suitable for the length of the duct to be inspected and able to be between 5 cm and 3 m. The insertion tube 3 has various shapes of cross section such as square, oval or circular. This insertion tube 3 which is in contact with the tissues, human organs or medical appliances (trochars or probes), essentially lies within the category of single or multiple use on one patient or even of re-usable use after decontamination, disinfection or sterilization.

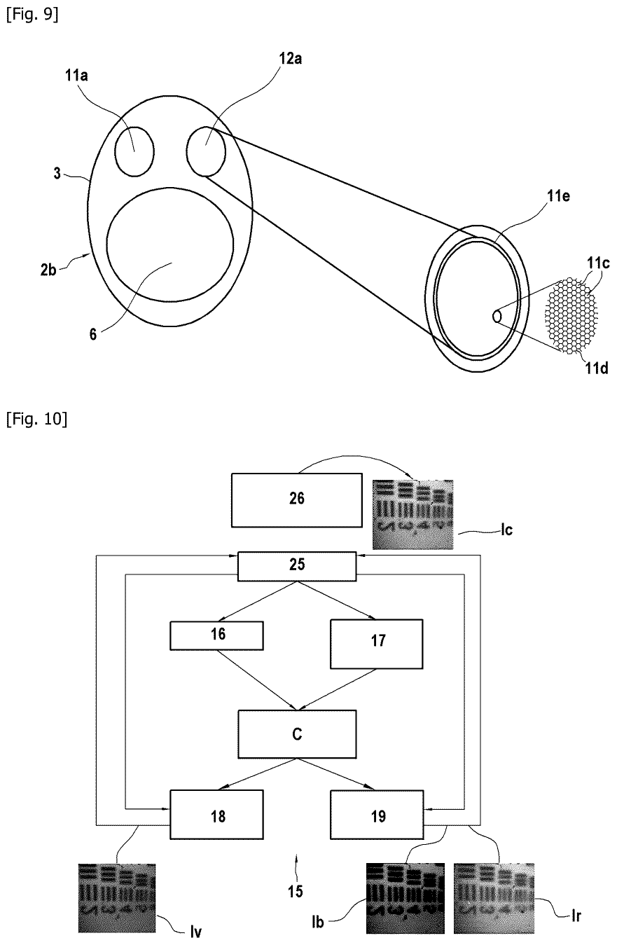

The endoscopic system 1 of endoscope type also includes inside the insertion tube 3 a tubular duct 6 forming an operating or work channel extending from the control handle 4 all the way to the distal head 2b to allow at this distal head, the entry of various tools and/or delivery of fluids and/or suction of fluids (FIG. 9). The tubular duct 6 is surrounded by the insertion tube 3 over its entire length between the distal head 2b and the control handle 4. Conventionally, the tubular duct 6 extends beyond the endpiece 3a inside the control handle 4.

Conventionally, the endoscopic system 1 of endoscope type also includes a control mechanism 8 making it possible to orient the distal head 2b with respect to the longitudinal axis of the insertion tube 3. For this purpose, the insertion tube 3 includes, upstream of the distal head 2b, a flexible, bending or articulating structure 9 allowing the orientation of the distal head 2b with respect to the longitudinal axis of the insertion tube 3. The control mechanism 8 can be embodied in any appropriate manner such that the distal head 2b can be displaced between an idle position in which the insertion tube 3 is rectilinear and articulated position in which the articulating part 9 is curved. For example, the control mechanism 8 may include a manual control lever rotationally driving a pulley to which is attached at least one actuating cable mounted to be attached at the distal head 2b.

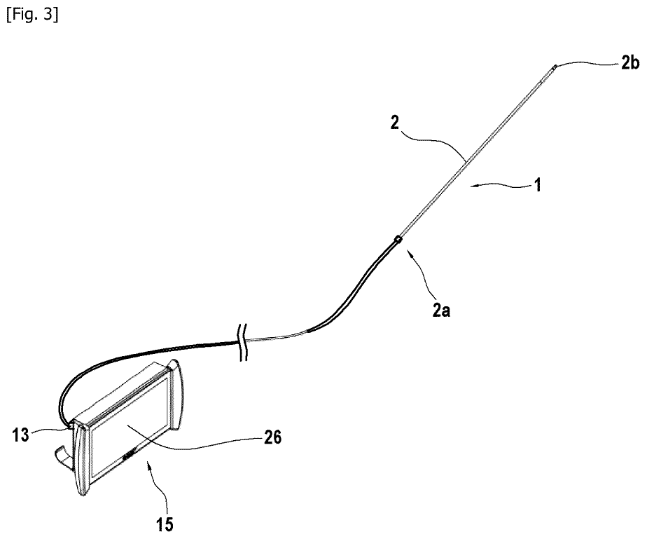

According to another mode of application for which the medical endoscopic system 1 is a catheter (FIG. 3), the medical endoscopic system includes as the insertion instrument 2 a catheter terminating opposite a proximal part 2a in a distal head 2b. Said catheter may be of conventional design with no actuating system making it possible to move the distal part along one or more axes. It can also be equipped with a deflection system with return to position by a shape memory structure, such as for example a plate or a thread made of nitinol.

Another actuating device of the distal part can be embodied by means of cables, of deformable parts by making use of the elasticity of the materials.

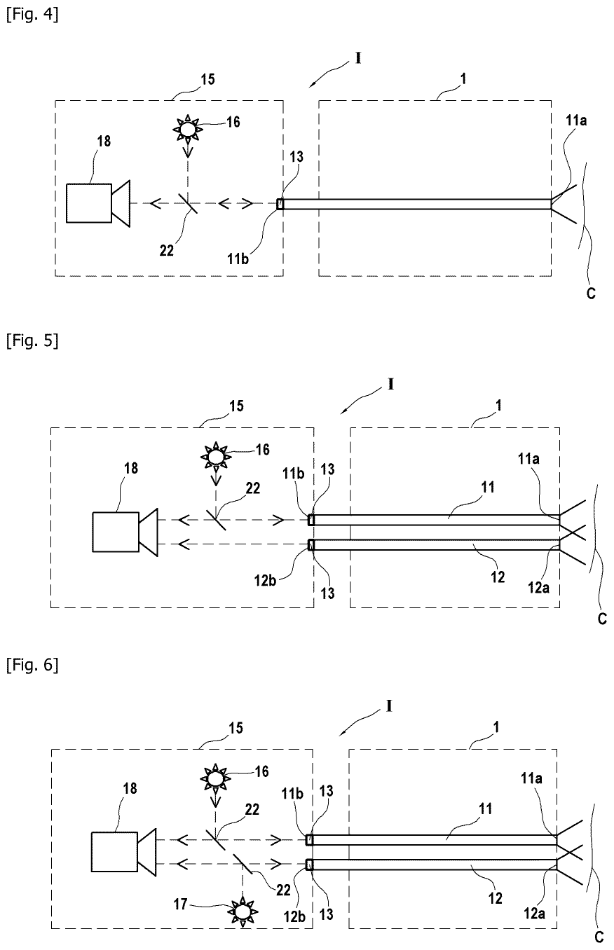

In accordance with the invention, the insertion instrument 2 is provided with at least one multicore optical fiber 11 as in the variant illustrated in FIG. 4 and a first multicore optical fiber 11 and a second multicore optical fiber 12 as in the variants illustrated in FIGS. 5 to 8. Each multicore optical fiber 11, 12 respectively has a distal end 11a, 12a and a proximal end 11b, 12b. The distal end 11a, 12a of the multicore optical fibers is located at the distal head 2b of the insertion instrument 2 in such a way as to view the target C. Note that an optical structure may be placed at the distal end 11a, 12a of the multicore optical fibers. Each multicore optical fiber 11, 12 extends at least all the way to the proximal part 2a of the insertion instrument and is provided with at least one optical connector 13 at its proximal end 11b, 12b.

Of course, the optical connector 13 equipping the proximal end 11b, 12b of the multicore optical fibers 11, 12 is intended to interact with a complementary male or female optical connector according to the female or male type of the optical connector 13. Advantageously, a focusing lens is mounted in the complementary connector making it possible to improve the optical connection, offering a wider positioning tolerance. Specifically, the optical connector 13 may be intended to be disposed of as waste with the insertion instrument. In this case, the optical connector 13 can be embodied economically with significant allowances for tolerance.

In a known manner, a multicore optical fiber 11, 12 is an optical fiber including a multitude of cores 11c (FIG. 9), for example at least 10 000 cores separated by a common cladding or a separating structure 11d such as a matrix. These cores 11c clad with the separating structure 11d are mounted inside a shared protecting sheath 11e. This structure 11d for separating the cores from one another has, along the section of the multicore optical fiber, a honeycomb shape. For example, the following can be used as multicore optical fibers 11, 12: optical fibers marketed under the trade name ESKA by the Mitsubishi Rayon Co. company, MBI by the Asahi Kasei company or else FIGP by the Fujikura company.

The imaging system I also include a device 15 for acquiring and processing images including either a single illumination source 16 as in the variants illustrated in FIGS. 4, 5, 7 or a first illumination source 16 and a second illumination source 17 as in the variants illustrated in FIGS. 6 and 8. Each illumination source 16, 17 is configured to deliver a light beam in at least a first wavelength spectrum, to a multicore optical fiber 11, 12 via the optical connector 13. Each illumination source 16, 17 is embodied in any appropriate way to allow the multicore optical fiber 11, 12, to deliver to its distal end 11a, 12a a light beam suitable for illuminating the target C to be imaged. For example, the illumination sources 16, 17 can be embodied by light-emitting diodes, halogen lamps, or infrared or ultraviolet radiation light sources.

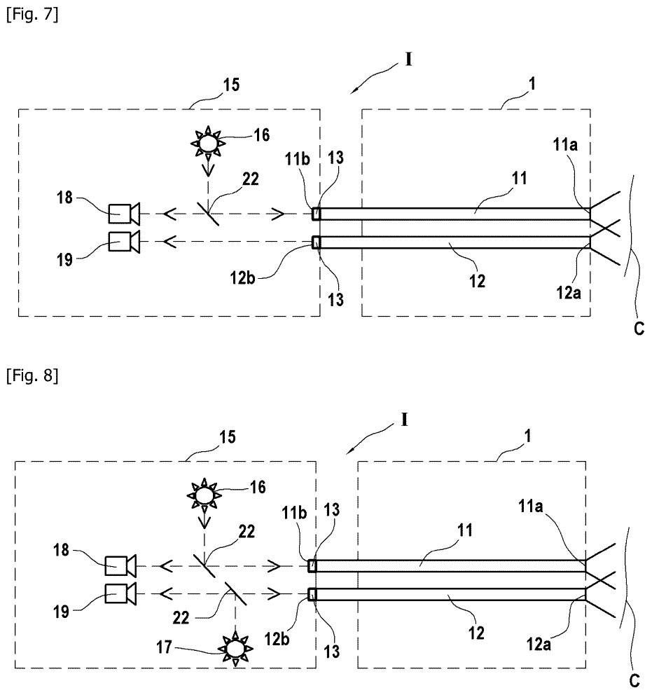

The device 15 for acquiring and processing images also includes either at least one imaging sensor 18 as in the variant embodiments illustrated in FIGS. 4, 5, 6 or else a first imaging sensor 18 and a second imaging sensor 19 as in the variant embodiments illustrated in FIGS. 7 and 8. Each imaging sensor 18, 19 is configured to receive a light beam coming from the proximal end of a multicore optical fiber 11, 12, each equipped with the optical connector 13.

As can be seen from the different variant embodiments, it should be noted that the illumination sources 16, 17 and the imaging sensors 18, 19 are part of the device 15 for acquiring and processing images and are thus remote from the medical endoscopic system 1. It ensues that in the case where the medical endoscopic system 1 is of disposable type, the illumination sources 16, 17 and the imaging sensors 18, 19 can be reused with another medical endoscopic system 1, thus reducing the electronic waste. Moreover, in the scenario where the medical endoscopic system 1 requires a decontamination operation, the device 15 for acquiring and processing images is not concerned by such an operation so that the illumination sources 16, 17 and the imaging sensors 18, 19 which are part of this device are not liable to be damaged by this decontamination operation.

Note that in the example of an application illustrated in FIG. 1 for which an endoscope is used as the medical endoscopic system 1, the proximal end of the multicore optical fiber or fibers, provided with the optical connector 13, is located at the proximal part 2a of the insertion instrument i.e. at the control handle 4.

The multicore optical fiber or fibers 11, 12 are mounted inside the insertion tube 2 but outside the tubular duct 6. The multicore optical fiber or fibers 11, 12 thus extend from the distal head of the insertion tube, being inserted over the entire length of the insertion tube 3, between this latter and the tubular duct 6. The multicore optical fiber or fibers 11, 12 extend inside the control handle 4 all the way to one or more optical connectors 13 mounted at the proximal part of the control handle 4.

According to this example, an optical cable 21 provides an optical link between the optical connector or connectors 13 and the device 15 for acquiring and processing images to convey the light beams between on the one hand, the multicore optical fiber or fibers 11, 12 and on the other hand, the illumination sources 16, 17 and the imaging sensor or sensors 18, 19. The optical cable 21 can be embodied in any appropriate way in the form of one or more optical fibers. Typically, the optical cable 21 is provided opposite its part connected to the optical connector 13, with an optical connector 13a attached to the device 15 for acquiring and processing images.

Note that in the example of an application illustrated in FIG. 2 for which an endoscope is used as the medical endoscopic system 1, the proximal end of the multicore optical fiber or fibers, provided with the optical connector 13 is linked directly to the device 15 for acquiring and processing images. According to this example, the proximal end of the multicore optical fiber or fibers, provided with the optical connector 13, is attached directly to the device 15 for acquiring and processing images. Thus, the multicore optical fiber or fibers 11, 12 extend from the distal head of the insertion tube, being inserted over the entire length of the insertion tube 3, between this latter and the tubular duct 6. The multicore optical fiber or fibers 11, 12 extend inside the control handle 4 to come out of the proximal part of the control handle in such a way as to be connected to the optical connector 13 attached to the device 15 for acquiring and processing images.

Note that in the example of an application illustrated in FIG. 3 for which a catheter is used as the medical endoscopic system 1, this catheter is formed at least by the multicore optical fiber or fibers 11, 12, the distal end 11a, 12a of which forms the distal head 2b of the insertion instrument 2. In the scenario where only one multicore optical fiber is used, the insertion instrument 2 is formed by this multicore optical fiber which can be built into or surrounded by a protective sleeve. In the scenario in which two multicore optical fibers 11, 12 are used, the insertion instrument 2 is formed by a protective sleeve in which the two multicore optical fibers 11, 12 are mounted. Note that the proximal end of the multicore optical fiber or fibers 11, 12 is linked via the optical connector 13, directly to the device 15 for acquiring and processing images (as illustrated on FIG. 3) or indirectly using the optical cable 21 (as explained in relation to FIG. 1).

According to a first exemplary embodiment illustrated on FIG. 4, the medical endoscopic system 1 includes a single multicore optical fiber 11 while the device 15 for acquiring and processing images includes:
an illumination source 16 configured to deliver a light beam in at least a first wavelength spectrum, to the single multicore optical fiber 11 via the optical connector 13;
a single imaging sensor 18 configured to receive a light beam coming from the proximal end of the single multicore optical fiber 11.

The device 15 for acquiring and processing images includes an optical separating system 22 disposed on the optical path between the proximal end of the multicore optical fiber 11 and the imaging sensor 18 and reflecting, in the direction of the proximal end of the multicore optical fiber 11, the light beam coming from the illumination source 16. This optical separating system 22 can be embodied by any appropriate means such as by a half-wave plate, a beam splitter or an optical prism system.

One advantage of this exemplary embodiment is of being able to precisely illuminate the area of the target observed by the imaging sensor and to minimize the diameter of the insertion instrument while reducing the waste generated by using only a single multicore optical fiber.

According to a second exemplary embodiment illustrated on FIG. 5, the endoscopic system 1 includes a first multicore optical fiber 11 and a second multicore optical fiber 12. The device 15 for acquiring and processing images includes:
an illumination source 16 configured to deliver a light beam to the first multicore optical fiber 11,
a single imaging sensor 16 configured to have a first area for receiving a light beam coming from the proximal end of the first multicore optical fiber 11 and a second receiving area separate from the first receiving area, for receiving a light beam coming from the proximal end of the second multicore optical fiber 12.

The device 15 for acquiring and processing images includes an optical separating system 22 disposed on the optical path between the proximal end of the multicore optical fiber 11 and the imaging sensor 18 and reflecting, in the direction of the proximal end of the multicore optical fiber 11, the light beam coming from the illumination source 16.

This exemplary embodiment has the advantage of being able to obtain two images simultaneously which may be processed at the same time to make an super-resolution as will be described in the remainder of the description.

According to a third exemplary embodiment illustrated in FIG. 6, the endoscopic system 1 includes a first multicore optical fiber 11 and a second multicore optical fiber 12. The device 15 for acquiring and processing images includes:
a first illumination source 16 configured to deliver a light beam to the first multicore optical fiber 11,
a second illumination source 17 configured to deliver a light beam to the second multicore optical fiber 12,
a single imaging sensor 18 configured to have a first area for receiving a light beam coming from the proximal end of the first multicore optical fiber 11 and a second receiving area separate from the first receiving area, for receiving a light beam coming from the proximal end of the second multicore optical fiber 12.

The device 15 for acquiring and processing images includes an optical separating system 22 disposed on the optical path between the proximal end of each multicore optical fiber 11, 12 and the imaging sensor 18 and reflecting, in the direction of the proximal end of each multicore optical fiber 11, 12, the light beam coming from the illumination source 16.

This exemplary embodiment makes it possible to illuminate the target with light beams having different wavelength spectra in order to obtain a spectral super-resolution image. This solution offers the advantage of being able to view tumors. Specifically, by choosing a specific wavelength spectrum, the vascularization of the tissues can be highlighted. As a tumor is a highly vascularized area, a tumor can be more closely observed by implementing this technique.

According to a fourth exemplary embodiment illustrated on FIG. 7, the endoscopic system 1 includes a first multicore optical fiber 11 and a second multicore optical fiber 12. The device 15 for acquiring and processing images includes:
an illumination source 16 configured to deliver a light beam to the first multicore optical fiber 11,
a first imaging sensor 18 configured to receive a light beam coming from the proximal end of the first multicore optical fiber 11,
a second imaging sensor 19 configured to receive a light beam coming from the proximal end of the second multicore optical fiber 12.

The device 15 for acquiring and processing images includes an optical separating system 22 disposed on the optical path between the proximal end of the multicore optical fiber 11 and the imaging sensor 18 and reflecting, in the direction of the proximal end of the multicore optical fiber 11, the light beam coming from the illumination source 16.

This example makes it possible to achieve a super-resolution insofar as it is possible to acquire two images on two imaging sensors. It is also possible to acquire the images one after the other with different wavelengths.

According to a fifth exemplary embodiment illustrated on FIG. 8, the endoscopic system 1 includes a first multicore optical fiber 11 and a second multicore optical fiber 12. The device 15 for acquiring and processing images includes:
a first illumination source 16 configured to deliver a light beam to the first multicore optical fiber 11,
a second illumination source 17 configured to deliver a light beam to the second multicore optical fiber 12,
a first imaging sensor 18 configured to receive a light beam coming from the proximal end of the first multicore optical fiber 11,
a second imaging sensor 19 configured to receive a light beam coming from the proximal end of the second multicore optical fiber 12.

The device 15 for acquiring and processing images includes an optical separating system 22 disposed on the optical path between the proximal end of each multicore optical fiber 11, 12 and the imaging sensor 18, 19 and reflecting, in the direction of the proximal end of each multicore optical fiber 11, 12, the light beam coming from the illumination sources 16, 17.

According to this example, it is possible to obtain images with twice as much resolution. One advantage of this solution is to be able to view tumors.

Note that on FIGS. 4 to 8, the proximal end of the multicore optical fiber or fibers, provided with the optical connector 13, is schematically represented as being directly connected to the device 15 for acquiring and processing images. Of course, the proximal end of the multicore optical fiber or fibers 11, 12, provided with the optical connector 13, can be located at the proximal part 2a of the insertion instrument such that an optical cable 21 provides the optical link between the optical connector 13 attached to the control handle 4 and the device 15 for acquiring and processing images.

In the same way, it must be considered that the device 15 for acquiring and processing images is configured in such a way as to convey the light between the imaging sensors 18, 19 and the optical connectors 13, 13a attached to the device 15 for acquiring and processing images. Similarly, the device 15 for acquiring and processing images is configured in such a way as to convey the light by any appropriate means between the illumination sources 16, 17 and the optical connectors 13, 13a attached to the device 15 for acquiring and processing images.

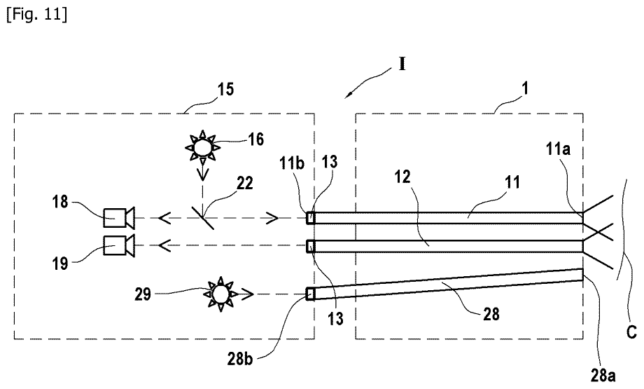

Note that according to FIGS. 4 to 8, the multicore optical fibers 11, 12 in particular convey the luminous flux from the illumination sources all the way to the distal head 2b of the insertion instrument. Note, as illustrated on FIG. 11, that it may be envisioned for the medical endoscopic system 1 to include an illuminating optical fiber 28 allowing the delivery of an additional luminous flux.

This illuminating optical fiber 28 has a distal end 28a and a proximal end 28b collecting the luminous flux from a light source 29. The distal end 28a of the illuminating optical fiber 28 is located at the distal head 2b of the insertion instrument while the proximal end of the illuminating optical fiber is located at the proximal part 2a of the insertion instrument while being provided with an optical connector through which is conveyed an illuminating light beam supplied by the light source 29. This illuminating optical fiber 28 can be implemented in all the exemplary embodiments described in this application.

The device 15 for acquiring and processing images also includes, as illustrated on FIG. 10, an imaging processor 25 linked to the imaging sensors 18, 19 and configured to form images of the target C, based on the signals delivered by the imaging sensors 18, 19. The imaging processor 25 controls the imaging sensors 18, 19 to acquire the images of the target at given times. The imaging processor 25 also controls the illumination sources 16, 17 to control the illumination emitted, particularly during the acquisition of the images by the imaging sensors 18, 19 as described in the remainder of the description. The imaging processor 25 is connected to a viewing screen 26 used to display the images of the target C. This viewing screen 26 can be part of the device 15 for acquiring and processing images or be remote from this device. Of course, the imaging processor 25 can be connected to a memory to record the images.

The device 15 for acquiring and processing images can take different forms. Conventionally, the device 15 for acquiring and processing images can take the form of an electronic tablet provided with the viewing screen 26 and with a human-machine interface allowing a user to enter data or to control this device. This human-machine interface can be a keyboard, a mouse, or the screen for example embodied by a touch-sensitive screen. The device 15 for acquiring and processing images also includes a communication unit configured to communicate with a database, generally remote, forming part of a computer system.

The imaging sensor I according to the invention can be implemented in different ways which arise directly from the preceding description.

According to an exemplary implementation, the illumination source or sources 16, 17 are configured to deliver light beams in different wavelength spectra and the imaging sensor or sensors 18, 19 are suitable for acquiring images of different wavelength spectra. Typically, it may be envisioned to acquire images with different acquisition times before reconstructing them.

Advantageously, the imaging processor 25 processes the images of different wavelength spectra to obtain a spectral super-resolution image. In other words, the resultant image has a greater resolution than the resolution of the images taken.

According to an advantageous exemplary embodiment, the illumination source or sources 16, 17 are configured to deliver light beams in the red, green and blue wavelength spectra and the imaging sensor or sensors 18, 19 are configured to acquire images in the red Ir, green Iv and blue Ib wavelength spectra. In the example illustrated on FIG. 10, the first illumination source 16 is controlled to deliver a light beam in a red wavelength spectrum and the first imaging sensor 18 is configured to acquire images in the red Ir wavelength spectrum. The second illumination source 17 is configured to successively deliver light beams in green and blue wavelength spectra and the second imaging sensor 19 is configured to acquire images in the green Iv and blue Ib wavelength spectra.

Moreover, the imaging processor 25 processes the wavelength spectrum images to obtain a contrasted or colored image Ic which can be a white image.

In the illustrated example, the imaging processor 25 processes the images in the red Ir, green Iv and blue Ib wavelength spectra to obtain a white image Ic.

Typically, for an imaging sensor 18, 19 of CMOS type with a BAYER matrix, each red Ir, green Iv and blue Ib wavelength spectrum for example has a resolution of 40 000 pixels. The inclusion of these images makes it possible to obtain a white image of a resolution of 120000 pixels.

According to another advantageous exemplary embodiment, the illumination source or sources 16, 17 are configured to successively deliver light beams in different wavelength spectra such infrared light radiation and ultraviolet light radiation.

According to another exemplary implementation, the imaging processor 25 controls the imaging sensor or sensors 18, 19 to acquire temporally-offset images. The imaging processor 25 processes the temporally-offset images to obtain a temporal super-resolution image. Thus, the imaging processor 25 processes a series of images taken successively over time in such a way as to obtain a resultant image with an improved resolution by comparison with the resolution of each image taken.

According to another exemplary implementation, the imaging processor 25 controls the imaging sensor or sensors 18, 19 to acquire images that are spatially offset while having an area of overlap. These images are spatially offset following the displacement of the insertion instrument 2 or given the offset of the two multicore optical fibers at the distal head 2b. The imaging processor 25 processes the spatially but also temporally offset images to obtain a spatial super-resolution image. Thus, the imaging processor 25 processes a series of images taken successively for different spatial positions of the distal head in such a way as to obtain a resultant image with an improved resolution by comparison with the resolution of each image taken.

The spatial, temporal and spectral super-resolution images are produced using image processing algorithms based on multi-image super-resolution methods. These methods are based on three different known approaches:

Interpolation Based approaches; Frequency domain-based approaches;

Reconstruction based approaches. These methods are briefly described in particular in the following publications: 1—S. Borman and R. Stevenson, Super-Resolution from Image Sequences: A Review, in Midwest Symposium on Circuits and Systems, Notre Dame, IN, USA, 8 1998, pp. 374-378. S. C. Park, M. K. Park, and M. G. Kang. 2—Super-Resolution Image Reconstruction: A Technical Overview, IEEE Signal Processing Magazine, vol. 20, No. 3, pp. 21-36, 5 200. 3—C. Mancas-Thillou and M. Mirmehdi, An Introduction to Super-Resolution Text, in Digital Document Processing, ser. Advances in Pattern Recognition. Springer London, 2007, pp. 305-327. 4—Tian and K.-K. Ma, A survey on super-resolution imaging, Signal, Image and Video Processing (SIViP), vol. 5, No. 3, pp. 329-342, 2011.

1. An imaging system for a medical endoscopic system for viewing a target including:
a medical endoscopic system including an insertion instrument terminating, opposite a proximal part, in a distal head, this insertion instrument being provided with at least one multicore optical fiber having a distal end and a proximal end, the distal end of the multicore optical fiber being located at the distal head of the insertion instrument while the multicore optical fiber is provided with an optical connector at its proximal end and extends at least all the way to the proximal part of the insertion instrument;
a device for acquiring and processing images including:
at least a first illumination source configured to deliver a light beam in at least a first wavelength spectrum, to the multicore optical fiber via the optical connector;
at least one imaging sensor configured to receive a light beam coming from at least the proximal end of the multicore optical fiber;
an imaging processor connected to the imaging sensor and configured to form images of the target.

2. The imaging system as claimed in claim 1 wherein the medical endoscopic system includes a single multicore optical fiber while the device for acquiring and processing images includes:
an illumination source configured to deliver a light beam in at least a first wavelength spectrum, to the single multicore optical fiber via the optical connector;
a single imaging sensor configured to receive a light beam coming from the proximal end of the single multicore optical fiber.

3. The imaging system as claimed in claim 1 wherein:
the endoscopic system includes:
a first multicore optical fiber and a second multicore optical fiber,
the device for acquiring and processing images includes:
an illumination source configured to deliver a light beam to the first multicore optical fiber,
a single imaging sensor configured to have a first area for receiving a light beam coming from the proximal end of the first multicore optical fiber and a second receiving area separate from the first receiving area, for receiving a light beam coming from the proximal end of the second multicore optical fiber.

4. The imaging system as claimed in claim 1 wherein:
the endoscopic system includes:
a first multicore optical fiber and a second multicore optical fiber,
the device for acquiring and processing images includes:
a first illumination source configured to deliver a light beam to the first multicore optical fiber,
a second illumination source configured to deliver a light beam to the second multicore optical fiber,
a single imaging sensor configured to have a first area for receiving a light beam coming from the proximal end of the first multicore optical fiber and a second receiving area separate from the first receiving area, for receiving a light beam coming from the proximal end of the second multicore optical fiber.

5. The imaging system as claimed in claim 1 wherein:
the endoscopic system includes:
a first multicore optical fiber and a second multicore optical fiber,
the device for acquiring and processing images includes:
an illumination source configured to deliver a light beam to the first multicore optical fiber,
a first imaging sensor configured to receive a light beam coming from the proximal end of the first multicore optical fiber,
a second imaging sensor configured to receive a light beam coming from the proximal end of the second multicore optical fiber.

6. The imaging system as claimed in claim 1 wherein:
the endoscopic system includes:
a first multicore optical fiber and a second multicore optical fiber
the device for acquiring and processing images includes:
a first illumination source configured to deliver a light beam to the first multicore optical fiber,
at least a second illumination source configured to deliver a light beam to the second multicore optical fiber,
a first imaging sensor configured to receive a light beam coming from the proximal end of the first multicore optical fiber,
a second imaging sensor configured to receive a light beam coming from the proximal end of the second multicore optical fiber.

7. The imaging system as claimed in claim 1 wherein:
the illumination source or sources are configured to deliver light beams in different wavelength spectra,
the imaging sensor or sensors are configured to acquire images of different wavelength spectra,
the imaging processor processes the images of different wavelength spectra to obtain a spectral super-resolution image.

8. The imaging system as claimed in claim 7, wherein the imaging sensor or sensors are configured to acquire images of different wavelength spectra by means of colored filters, the colors of which correspond to the different wavelength spectra of the light beams.

9. The imaging system as claimed in claim 7 wherein:
the illumination source or sources, are configured to deliver light beams in the red, green and blue wavelength spectra;
the imaging sensor or sensors, are configured to acquire images in the red, green and blue wavelength spectra;
the imaging processor processes the wavelength spectra images to obtain a contrasted or colored image.

10. The imaging system as claimed in claim 1 wherein:
the imaging processor controls the imaging sensor or sensors, to acquire temporally-offset images,
the imaging processor processes the temporally-offset images to obtain a temporal super-resolution image.

11. The imaging system as claimed in claim 10, wherein the insertion instrument is static.

12. The imaging system as claimed in claim 1 wherein:
the imaging processor controls the imaging sensor or sensors to acquire spatially-offset images having an area of overlap,
the imaging processor processes the spatially-offset images to obtain a spatial super-resolution image.

13. The system as claimed in claim 12, wherein the spatially-offset images with an area of overlap are obtained using two networks in the multicore fiber or fibers, these two networks being offset with respect to one another.

14. The imaging system as claimed in claim 1 wherein the medical endoscopic system includes an illuminating optical fiber having a distal end and a proximal end, the distal end of the illuminating optical fiber being located at the distal head of the insertion instrument while the proximal end of the illuminating optical fiber is located at the proximal part of the insertion instrument while being provided with an optical connector via which is conveyed an illuminating light beam supplied by an illumination source.

15. The imaging system as claimed in claim 1 wherein the device for acquiring and processing images includes an optical separating system disposed on the optical path between the proximal end of the multicore optical fiber and an imaging sensor and reflecting in the direction of the proximal end of the multicore optical fiber, the light beam coming from an illumination source.

16. The imaging system as claimed in claim 1 wherein the medical endoscopic system includes as the insertion instrument an insertion tube terminating in a distal head and held at its opposite end by a control handle (4), the multicore optical fiber or fibers being mounted inside the insertion tube.

17. The imaging system as claimed in claim 1 wherein the medical endoscopic system includes as the insertion instrument a catheter including the multicore optical fiber or fibers.
